(19) United States
(12) Patent Application Publication
(10) Pub. No.: US 2009/0239923 A1
(43) Pub. Date: Sep. 24, 2009

(54) CONCENTRATED AQUEOUS AND/OR ETHANOLIC SOLUTION OF 4-METHYLPYRAZOLE (FOMEPIZOLE)

(76) Inventor: Christopher N. Jobdevairakkam, Plainsboro, NJ (US)

Correspondence Address: ST. ONGE STEWARD JOHNSTON & REENS, LLC, 986 BEDFORD STREET, STAMFORD, CT, 06905-5619, US

(21) Appl. No.: 12/408,404

(22) Filed: Mar. 20, 2009

Related U.S. Application Data
(60) Provisional application No. 61/038,542, filed on Mar. 21, 2008.

(51) Int. Cl. A61K 31/415

(52) U.S. Cl. 514/406

(57) ABSTRACT

A composition of matter comprising fomepizole and water wherein the content of water is about 3% to about 40% by weight, and methods of making fomepizole solutions effective to reduce the freezing point of pure fomepizole to less than about 18° C.

### CROSS-REFERENCE TO RELATED APPLICATIONS

This application claims the benefit under 35 U.S.C. 119(e) of the U.S. Provisional Application No. 61/038,542, filed on Mar. 21, 2008.

### FIELD OF THE INVENTION

The present invention relates to a composition of fomepizole which does not cause freezing of fomepizole at room temperature, and even at temperatures below 0° C.

### BACKGROUND OF THE INVENTION

Fomepizole is chemically described as 4-methylpyrazole and is administered to treat a small population of patients who have ingested ethylene glycol or methanol either intentionally or accidentally, or canines who have ingested ethylene glycol. Ethylene glycol is a component in antifreeze liquids used in automobiles, coolants, and other thermal (heat-transfer) fluid applications. Methanol is used as an antifreeze component in windshield washer liquid. In third world countries, methanol poisoning is one of the most commonly cited medical emergencies. A 2005 data review of the American Poison Control Center's National Poison and Exposure Database, incorporated herein by reference, identified nearly 6,000 exposures to ethylene glycol which resulted in 16 deaths and 176 near fatalities (M. W. Lai et. al., Clinical Toxicology 2006, 44:803-932). Ingestion of 90 mL of methanol (70 g) in a 70 Kg human body is a recently cited figure as being a lethal dose (D. Jacobsen, and K. E. McMartin, “Methanol and ethylene glycol poisonings, mechanism of toxicity, clinical course, diagnosis and treatment,” Medical Toxicology, 1986, 1:309-334).

Accidental or intentional ingestion of ethylene glycol qualifies as a rare disorder (affecting fewer than 200,000 people) and therefore the Antizol brand (available from Jazz Pharmaceuticals, Palo Alto, Calif.) has been granted “orphan drug” status by the US FDA. This injectable dosage form of fomepizole is marketed in the US under the Brand Name “ANTIZOL” by Jazz Pharmaceuticals, and contains 1.5 mL of fomepizole per vial with no additives or solvents (i.e. undiluted). Fomepizole freezes at about 20° to 24° C. and, therefore, when vials are stored at room temperature, the fomepizole often solidifies or develops suspended solidified particulate matter. Accordingly, it is advised to melt the contents of the vial by holding under running warm water or by holding in one's hand, and only then withdrawing using a syringe. The fomepizole is diluted prior to administration (according to the packaging insert accompanying ANTIZOL brand injectable). The particular dosing directions are for one vial of Antizol to be diluted into at least 100 mL of 0.9% sodium chloride solution or dextrose 5% solution, and the resultant mixture administered over a period of 30 minutes. Fomepizole should never be given undiluted or by bolus injection because of the risk of venous irritation and phlebosclerosis. Since fomepizole is administered to patients who have ingested ethylene glycol or methanol and are often admitted under life-threatening emergency conditions, initiating treatment is very much critical to meeting the emergency treatment protocol. Because Antizol is mostly frozen at room temperature, or at least typically contains frozen particles, valuable time is wasted in preparing the injection by melting the frozen fomepizole vials and withdrawing the melted fomepizole into a syringe needle to withdraw the drug.

In addition to the fomepizole vial having to be warmed to liquefy the ingredient prior to dilution and administration, the melted fomepizole in the vial can solidify in the syringe needle used to withdraw if from the vial if the needle is also not warmed above the melting point; sterile syringes are kept at room temperature, typically about 20° C. to 25° C. The drawback to using a product like Antizol is the concurrent requirement for keeping the syringe at least above 25° C. so as to prevent the fomepizole from solidifying and thus clogging the needle. The possibility of fomepizole freezing in the syringe needle is certainly not a satisfactory operation, and can waste valuable time during an emergency treatment.

Further, fomepizole is reported to be soluble in water. In the pharmacological arts, “water soluble” in this context connotes that one portion of fomepizole should be soluble in 30 portions of water. The concentration of fomepizole in such solutions is about 3.3% v/v. Such dilute solutions are prone to instability due to microbial contamination. Consequently, the packaging insert of Antizol, incorporated herein by references, advises diluting one vial into 100 mL of saline or dextrose solution wherein the concentration of fomepizole is about 1.5% v/v, and to use that solution within 24 hours. While the protocol is also to store the diluted solution refrigerated or at room temperature, diluted solutions later showing haziness, particulates, a precipitate, or discoloration should not be used.

Because fomepizole is typically administered as part of an emergency treatment, the need to urgently prepare the injection preparation means there is still a need for a formulation that does not freeze at ambient temperature. There also is a need for specialized laboratory monitoring during administration (to check ethylene glycol or methanol concentrations, as well as the patient's blood pH, to avoid metabolic acidosis).

### SUMMARY OF THE INVENTION

According to the present invention, a novel composition of fomepizole and water which does not freeze and remains in liquid form above 0° C. is disclosed. The novel formulation allows the medical practitioner to provide the parenteral dosage in a vial or prefilled syringe without delay, in order to attend immediately to the medical emergency.

The present invention also discloses a novel composition of fomepizole that remains as liquid even at −6° C. and does not freeze even if stored in a refrigerator. The novel composition disclosed herein comprises fomepizole and intravenously acceptable liquids such as water and or ethanol. Also, the present invention reports a novel composition of fomepizole and water at an equimolar ratio that is suitable for parenteral administration.

In one embodiment, the present invention discloses a novel composition of matter comprising fomepizole and an intravenously acceptable liquid such as water and/or ethanol which does not cause freezing of fomepizole even at less than 0° C. The composition of fomepizole with equimolar water is mostly suitable for parenteral administration which does not freeze even at −6° C.

### DETAILED DESCRIPTION OF THE INVENTION

Fomepizole is currently available in vials as a neat liquid which can solidify at room temperature and usually requires warming to at least about 25° C. The possibility of fomepizole freezing in the syringe also requires that the syringe needle be at a warmed temperature; plugging of the syringe due to crystallization (freezing) exacerbates a situation during which life saving emergency treatment of a patient is occurring.

The present invention discloses a novel composition of matter comprising fomepizole and intravenously acceptable liquids such as water and/or ethanol. These compositions do not freeze even at less than 15° C. A composition of fomepizole with equimolar water is mostly suitable for parenteral administration and does not freeze even at −6° C. At an equimolar ratio of fomepizole and water, the freezing point is below −6° C. (and thus, while not desirous of being constrained to any particular theory, the equimolar ratio could be a monohydrate of fomepizole).

In the present invention, it is found that fomepizole is soluble in water but not miscible with water at all proportions. At a ratio of about 20 to 50% of fomepizole in water, the mixture is heterogeneous at room temperature (22° C.) but becomes a single phase upon warming to about 25° C., and upon cooling to about 22° C. the mixture turns back into a biphasic solution having separate layers. Such biphasic compositions are not suitable for making dosage preparations. Unexpectedly, at an approximately equimolar ratio of fomepizole to water, the mixture exhibits a freezing point of about −6° C. This equimolar ratio of fomepizole and water is most suitable for parenteral dosage form preparation. Thus, according one embodiment, this invention provides a pharmaceutical composition comprising fomepizole and water at an essentially equimolar ratio (within 10%, preferably 5% of being equimolar). A mixture of fomepizole and water in the molar ratio where in the fomepizole is between 50 to 90% freezes at or below 0° C. The mixture of about 82 g of fomepizole and about 18 g of water (equimolar) is freely miscible at room temperature and upon cooling to 0° C. the mixture is homogeneous. Upon further cooling to −6° C., crystallization without phase separation was observed.

The following examples are illustrative of embodiments of the present invention.

### EXAMPLE 1

Quantitatively, mixtures of various ratios of water and fomepizole were prepared. A general procedure for making the mixture of fomepizole and water is described below.

Into a glass cylinder of capacity 25 mL, fitted with a glass stirrer and a thermometer (range −50° C. to 110° C.), was added the weighed quantity of Fomepizole as directed in Table 1. To the same glass cylinder was added the weighed quantity of water as directed in Table 1. The mixture of fomepizole and water was manually stirred well using the glass stirrer and the temperature and appearance while at room temperature were noted. The glass cylinder with its content was placed in an ice-salt bath, stirred continuously and the appearance and the freezing point were observed. The measured freezing point and miscibility temperature for these mixtures are given in Table 1.

TABLE 1
Fomepizole composition with water

| | Wt | | | | | | |
|---|---|---|---|---|---|---|---|
| | of | Wt of | | | | Miscibility | Crystallisation/ |
| | water | Fomepizole | % | % | Appearance of | temp, | Freezing temp, |
| Exp # | (g) | (g) | water | Fomepizole | phase at 22° C. | ° C. | ° C. |
| A1 | 0.025 | 4.975 | 0.5 | 99.5 | Homogeneous, | NA | 22 |
| | | | | | clear solution | | |
| A2 | 0.05 | 4.95 | 1 | 99 | Homogeneous, | NA | 22 |
| | | | | | clear solution | | |
| A3 | 0.1 | 4.9 | 2 | 98 | Homogeneous, | NA | 20 |
| | | | | | clear solution | | |
| A4 | 0.15 | 4.85 | 3 | 97 | Homogeneous, | NA | 16 |
| | | | | | clear solution | | |
| A5 | 0.2 | 4.8 | 4 | 96 | Homogeneous, | NA | 11 |
| | | | | | clear solution | | |
| A6 | 0.25 | 4.75 | 5 | 95 | Homogeneous, | NA | 10 |
| | | | | | clear solution | | |
| A7 | 0.5 | 4.5 | 10 | 90 | Homogeneous, | NA | −4 |
| | | | | | clear solution | | |
| A8 | 0.75 | 4.25 | 15 | 85 | Homogeneous, | NA | −8 |
| | | | | | clear solution | | |
| A9 | 0.9 | 4.1 | 18 | 82 | Homogeneous, | NA | −9 |
| | | | | | clear solution | | |
| A10 | 1 | 4 | 20 | 80 | Homogeneous, | NA | −6 |
| | | | | | clear solution | | |
| A11 | 1.25 | 3.75 | 25 | 75 | Homogeneous, | NA | −3 |
| | | | | | clear solution | | |
| A12 | 2 | 3 | 40 | 60 | Homogeneous, | 18 | 0 |
| | | | | | clear solution | | |
| A13 | 2.5 | 2.5 | 50 | 50 | Hazy biphasic | 24 | 0 |
| | | | | | liquid | | |
| A14 | 3 | 2 | 60 | 40 | Hazy biphasic | 25 | 0 |
| | | | | | liquid | | |
| A15 | 3.5 | 1.5 | 70 | 30 | Hazy biphasic | 24 | 0 |
| | | | | | liquid | | |
| A16 | 4 | 1 | 80 | 20 | Hazy biphasic | 23 | 0 |
| | | | | | liquid | | |
| A17 | 4.5 | 0.5 | 90 | 10 | Homogeneous, | 0 | −1 |
| | | | | | clear solution | | |

Miscibility was determined visually (i.e., whether the solution appeared homogeneous and clear, or hazy).

According to another embodiment on this invention, a mixture of fomepizole and ethanol in the ratio where in ethanol is more than about 15% does not freeze at 0° C. The detailed freezing/crystallization temperature for various compositions of fomepizole and ethanol was determined. The results are given in Table 2.

### EXAMPLE 2

Quantitatively, mixtures of various ratios of Ethanol 200 (Aldrich) proof NF grade and fomepizole were prepared. A general procedure for making the mixture of fomepizole and ethanol is described below.

Into a glass cylinder of capacity 25 mL, fitted with a glass stirrer and a thermometer (range −50° C. to 110° C.), was added the weighed quantity of fomepizole as directed in Table 2. To the same glass cylinder was added the weighed quantity of ethanol as directed in Table 2. The mixture of fomepizole and ethanol was manually stirred well using the glass stirrer and the temperature and appearance while at room temperature were noted. The glass cylinder with its content was placed in an ice-salt-bath, stirred continuously and the appearance and the freezing point were observed. The measured freezing point and miscibility temperature for these mixtures are given in Table 2.

TABLE 2
Fomepizole compositions with ethanol

| | Wt of | Wt of | | | | Crystallisation/ |
|---|---|---|---|---|---|---|
| | water | Fomepizole | % | % | Appearance of | Freezing |
| Exp # | (g) | (g) | Ethanol | Fomepizole | phase at 22° C. | temp, ° C. |
| B1 | 0.0395 | 3.95 | 1.0 | 99.0 | Homogeneous, clear | 21 |
| | | | | | solution | |
| B2 | 0.1975 | 3.95 | 4.8 | 95.2 | Homogeneous, clear | 18 |
| | | | | | solution | |
| B3 | 0.3555 | 3.95 | 8.3 | 91.7 | Homogeneous, clear | 9 |
| | | | | | solution | |
| B4 | 0.6715 | 3.95 | 14.5 | 85.5 | Homogeneous, clear | −1 |
| | | | | | solution | |
| B5 | 0.8295 | 3.95 | 17.4 | 82.6 | Homogeneous, clear | −6 |
| | | | | | solution | |
| B6 | 0.9875 | 3.95 | 20.0 | 80.0 | Homogeneous, clear | −7 |
| | | | | | solution | |
| B7 | 1.1455 | 3.95 | 22.5 | 77.5 | Homogeneous, clear | −9 |
| | | | | | solution | |
| B8 | 1.1455 | 2.55 | 31.0 | 69.0 | Homogeneous, clear | below −10 |
| | | | | | solution | |
| B9 | 1.9355 | 2.55 | 43.2 | 56.8 | Homogeneous, clear | below −10 |
| | | | | | solution | |
| B10 | 2.7255 | 2.55 | 51.7 | 48.3 | Homogeneous, clear | below −10 |
| | | | | | solution | |
| B11 | 3.5155 | 2.55 | 58.0 | 42.0 | Homogeneous, clear | below −10 |
| | | | | | solution | |
| B12 | 5.0955 | 2.55 | 66.6 | 33.4 | Homogeneous, clear | below −10 |
| | | | | | solution | |

According to another embodiment of this invention, in a mixture of fomepizole, water, and ethanol in the ratio where in the content of ethanol is at least 0.1% by weight, the mixture does not freeze at 15° C. The miscibility and crystal formation temperatures for various mixtures of fomepizole, water, and ethanol were determined. The results are given in Table 3.

### EXAMPLE 3

Quantitatively, mixtures of various ratios of water, Ethanol 200 (Aldrich) proof NF grade and fomepizole were prepared. A general procedure for making the mixture of fomepizole, ethanol and water is described below.

Into a glass cylinder of capacity 25 mL, fitted with a glass stirrer and a thermometer (range −50° C. to 110° C.), was added the weighed quantity of fomepizole as directed in Table 3. To the same glass cylinder was added the weighed quantity of ethanol and water as directed in Table 3. The mixture of fomepizole, ethanol and water was manually stirred well using the glass stirrer and the temperature and appearance while at room temperature were noted. The glass cylinder with its content was placed in an ice-salt bath, stirred continuously and the appearance and the freezing point were observed. The measured freezing point and miscibility temperature for these mixtures are given in Table 3.

TABLE 3
Fomepizole solution with water and ethanol

| | Wt of | Wt of | Wt Of | | | | | | Crystallisation/ |
|---|---|---|---|---|---|---|---|---|---|
| | water | Fomepizole | Ethanol | % | % | % | Appearance of phase | Miscibility | Freezing |
| Exp # | (g) | (g) | (g) | water | Ethanol | Fomepizole | at 22° C. | temp, ° C. | temp, ° C. |
| A12 | 2 | 3 | 0.00 | 40.00 | 0.00 | 60.00 | Clear Homogeneous solution | 18 | 0 |
| C-1 | 2 | 3 | 0.08 | 40.80 | 0.14 | 59.07 | Clear Homogeneous solution | 9 | 0 |
| C-2 | 2 | 3 | 0.16 | 41.59 | 0.24 | 58.16 | Clear Homogeneous solution | 4 | 0 |
| C-3 | 2 | 3 | 0.32 | 43.08 | 0.48 | 56.43 | Clear Homogeneous solution | −7 | below −9 |
| A13 | 2.5 | 2.5 | 0.00 | 50.00 | 0.00 | 50.00 | Hazy biphasic liquid | 24 | 0 |
| C-4 | 2.5 | 2.5 | 0.08 | 50.66 | 0.12 | 49.22 | Clear Homogeneous solution | 15 | 0 |
| C-5 | 2.5 | 2.5 | 0.16 | 51.32 | 0.21 | 48.47 | Clear Homogeneous solution | 5 | −2 |
| C-6 | 2.5 | 2.5 | 0.24 | 51.95 | 0.31 | 47.74 | Clear Homogeneous solution | 0 | −6 |
| C-7 | 2.5 | 2.5 | 0.32 | 52.56 | 0.41 | 47.03 | Clear Homogeneous solution | −4 | below −9 |
| A14 | 3 | 2 | 0.00 | 60.00 | 0.00 | 40.00 | Hazy biphasic liquid | 25 | 0 |
| C-8 | 3 | 2 | 0.08 | 60.53 | 0.09 | 39.38 | Clear Homogeneous solution | 17 | 0 |
| C-9 | 3 | 2 | 0.24 | 61.52 | 0.29 | 38.19 | Clear Homogeneous solution | 5 | Below −9 |
| C-10 | 3 | 2 | 0.40 | 62.43 | 0.49 | 37.07 | Clear Homogeneous solution | below −7 | Below −9 |
| A15 | 3.5 | 1.5 | 0.00 | 70.00 | 0.00 | 30.00 | Hazy biphasic liquid | 24 | 0 |
| C-11 | 3.5 | 1.5 | 0.16 | 70.75 | 0.17 | 29.08 | Clear Homogeneous solution | 10 | 0 |
| C-12 | 3.5 | 1.5 | 0.32 | 71.44 | 0.34 | 28.22 | Clear Homogeneous solution | 0 | below −9 |
| C-13 | 3.5 | 1.5 | 0.47 | 63.12 | 9.48 | 27.40 | Clear Homogeneous solution | −7 | below −9 |
| A16 | 4 | 1 | 0.00 | 80.00 | 0.00 | 20.00 | Hazy biphasic liquid | 23 | 0 |
| C-14 | 4 | 1 | 0.16 | 77.45 | 3.16 | 19.39 | Clear Homogeneous solution | 6 | below −9 |
| C-15 | 4 | 1 | 0.32 | 74.87 | 6.32 | 18.81 | Clear Homogeneous solution | −4 | below −9 |
| C-16 | 4 | 1 | 0.47 | 72.25 | 9.48 | 18.27 | Clear Homogeneous solution | below −7 | below −9 |
| A17 | 4.5 | 0.5 | 0.00 | 90.00 | 0.00 | 10.00 | Clear Homogeneous solution | 0 | −1 |
| C-17 | 4.5 | 0.5 | 0.16 | 87.15 | 3.16 | 9.69 | Clear Homogeneous solution | below −7 | below −9 |

The pharmaceutical composition disclosed in this invention can be administered either by an oral route or a parenteral route, and can be administered during hemodialysis when necessary in certain cases of ethylene glycol poison treatment where more toxic by-products are observed in the blood (tending to require removal by dialysis). The most suitable means of administration is parenteral, although other means of administration known in the art may also be employed. Packaging and containers known in the field may be employed. For example, and not by way of limitation, a pharmaceutical composition of the present invention may be placed in sealed vials. Optionally, the sealed vials may be steam sterilized.

### EXAMPLE 4

About 1640 g of pure Fomepizole was weighed and transferred into a clean glass flask fitted with a mechanical stirrer and thermometer. The temperature of the flask was maintained at about 25° C. About 360 g of water for injection was added and stirred to mix for about 15 minutes. The mixture was tested for water content. Observed water content=18.4%. The mixture was filtered through 0.2μ filter and filled into vials and sealed. The fomepizole-water mixture prepared by this Example may be further diluted with a physiologically acceptable medium prior to administration.

Optionally, a composition of the present invention may include one or more of the following: stabilizers, solubilizers, buffers dispersing agents or flavoring agents. Examples of optional components include intravenously acceptable liquids, dextrose solution, sodium chloride solution, buffer solutions, carrier solutions, diluents, solvent. Examples of optional components also include dextrose, sodium chloride, polysorbate, carboxymethylcellulose, acacia, povidone, gelatin, sorbitol, citric acid, tartaric acid, EDTA, pluronic F-68 and phospholipids For a more extensive list, refer to Encyclopedia of Pharmaceutical Technology, Volume 19 (J. Swarbick and J. C. Boylan, 2000), herein incorporated by reference.

In an embodiment of the present invention, a composition of matter comprising fomepizole and water wherein the content of water is about 3% to about 40% by weight is disclosed. In another embodiment of the present invention, a composition of matter comprising fomepizole and water wherein the content of water is about 3% to about 40% by weight is disclosed and the composition is effective to reduce the freezing point of pure fomepizole to less than about 18° C. In yet another embodiment of the present invention, a composition of matter comprising fomepizole and water wherein the content of water is about 3% to about 40% by weight is disclosed, which composition is effective to reduce the freezing point of pure fomepizole to less than about 18° C., and wherein the amount of water is effective to reduce the freezing point to less than about −6° C.

Another embodiment of the present invention includes a composition of matter comprising fomepizole and water wherein the content of water is about 3% to about 40% by weight, which composition comprises about 18% by weight water and about 82% by weight fomepizole. An embodiment of the present invention may include an amount of water that is effective to reduce the freezing point to less than about −6° C.

In another embodiment of the present invention, a composition of matter comprising fomepizole and ethanol wherein the amount of ethanol is about 4% or more by weight is disclosed. In yet another embodiment of the present invention, a composition of matter comprising fomepizole and ethanol wherein the amount of ethanol is about 4% or more by weight is disclosed and the amount of ethanol is effective to reduce the freezing point of pure fomepizole to less than about 18° C.

In yet another embodiment, the present invention includes a composition of matter comprising fomepizole, water, and ethanol, wherein the content of ethanol is about 0.1% by weight. In yet another embodiment of the present invention, a composition of matter comprising fomepizole, water, and ethanol, wherein the content of ethanol is about 0.1% by weight is disclosed and the combined amounts of water and ethanol are effective to reduce the freezing point of pure fomepizole to less than about 18° C.

In any of the above embodiments, the water may be present at an equimolar ratio to the fomepizole.

Further disclosed is a method for making an injectable solution of fomepizole, comprising the steps of (a) providing a concentrated solution comprising fomepizole and at least one of water or ethanol effective to reduce the freezing point of pure fomepizole to less than about 18° C., and (b) diluting the concentrated solution with water containing sodium chloride or dextrose effective to make an injectable solution.

Another method is disclosed for making a concentrated solution of fomepizole having a reduced freezing point, comprising admixing fomepizole with ethanol, and at least 3% by weight of water, or a combination thereof. Other methods embodied by the present invention include methods of making a concentrated solution of fomepizole having a reduced freezing point, comprising admixing fomepizole with ethanol, and at least 3% by weight of water, or a combination thereof, in which method the freezing point is reduced to about 18° C. or about 0° C. or about −6° C. or even about −9° C.

Fomepizole used for these examples was synthesized by the synthetic procedure known in the art (Daniel L Reger et al., New. J. Chem. 2003, 27:1670-1677; Rita Menicagli, et al., Tetrahedron 1987, 43:171-177).

The foregoing description is meant to be illustrative and not limiting. Various changes, modifications, and additions may become apparent to the skilled artisan upon a perusal of this specification, and such are meant to be within the scope and spirit of the invention as defined by the claims.

What is claimed is:

1. A composition of matter comprising fomepizole and water wherein the content of water is about 3% to about 40% by weight.

2. A composition of matter comprising fomepizole and ethanol wherein the amount of ethanol is about 4% or more by weight.

3. A composition of matter comprising fomepizole, water, and ethanol, wherein the content of ethanol is about 0.1% by weight.

4. The composition of claim 1 containing about 18% by weight water and about 82% by weight fomepizole.

5. The composition of claim 1 wherein the water is present at an equimolar ratio to the fomepizole.

6. The composition of claim 1, wherein the amount of water is effective to reduce the freezing point of pure fomepizole to less than about 18° C.

7. The composition of claim 2, wherein the amount of ethanol in the mixture is effective to reduce the freezing point of pure fomepizole to less than about 18° C.

8. The composition of claim 3, wherein the combined amounts of water and ethanol are effective to reduce the freezing point of pure fomepizole to less than about 18° C.

9. The composition of claim 3 wherein the water is present at an equimolar ratio to the fomepizole.

10. The composition of claim 4 wherein the water is present at an equimolar ratio to the fomepizole.

11. The composition of claim 6, wherein the amount of water is effective to reduce the freezing point to less than about −6° C.

12. A method for making an injectable solution of fomepizole, comprising the steps of (a) providing a concentrated solution comprising fomepizole and at least one of water or ethanol effective to reduce the freezing point of pure fomepizole to less than about 18° C., and (b) diluting the concentrated solution with water containing sodium chloride or dextrose effective to make an injectable solution.

13. A method for making a concentrated solution of fomepizole having a reduce freezing point, comprising admixing fomepizole with ethanol, and at least 3% by weight of water, or a combination thereof.

14. The method of claim 13, wherein the freezing point is reduced to about 18° C.

15. The method of claim 14, wherein the freezing point is reduced to about 0° C.

16. The method of claim 14, wherein the freezing point is reduced to about −6° C.

17. The method of claim 14, wherein the freezing point is reduced to about −9° C.
